(19) United States
(12) Patent Application Publication
(10) Pub. No.: US 2007/0225339 A1
(43) Pub. Date: Sep. 27, 2007

(54) Compositions and methods for treating rheumatoid arthritis

(76) Inventors: Ramon Mohanlal, Boston, MA (US); Robert Kauffman, Chestnut Hill, MA (US); John Alam, Cambridge, MA (US); Christopher Godfrey, Natick, MA (US); Irina Kadiyala, Newton, MA (US)

Correspondence Address: VERTEX PHARMACEUTICALS INC., 130 WAVERLY STREET, CAMBRIDGE, MA, 02139-4242, US

(21) Appl. No.: 11/715,028

(22) Filed: Mar. 7, 2007

Related U.S. Application Data
(60) Provisional application No. 60/779,862, filed on Mar. 7, 2006.
(60) Provisional application No. 60/780,277, filed on Mar. 8, 2006.

(51) Int. Cl. A61K 31/44

(52) U.S. Cl. 514/352

(57) ABSTRACT

The present invention relates to methods and compositions for treating RA in subjects in need thereof. The invention also relates to kits and pharmaceutical packs comprising compositions and dosage forms.

This application claims the benefit of priority of U.S. Provisional Application 60/779,862, filed Mar. 7, 2006, and U.S. Provisional Application 60/780,277, filed Mar. 8, 2006, which are hereby incorporated by reference in their entirety.

### TECHNICAL FIELD OF THE INVENTION

The present invention relates to methods for treating rheumatoid arthritis.

### BACKGROUND OF THE INVENTION

Rheumatoid arthritis (RA) is characterized by chronic and progressive inflammatory processes in affected joints and systemic immunological abnormalities that leads to synovial hyperplasia and joint destruction. Cytokines that are abundantly produced in inflamed rheumatoid synovial fluid, such as tumor necrosis factor-α (TNF-α), interleukin-1β (IL-1β), IL-6 and IL-8, play crucial roles in the pathophysiology of RA. The significance of proinflammatory cytokines in the pathogenesis of RA is highlighted by the clinical effectiveness of specific inhibitors of TNF-α and IL 1β (Kremer, Rational use of new and existing disease-modifying agents in rheumatoid arthritis. Ann. Intern. Med. 134:695-706, 2001).

The treatment of RA has undergone a dramatic change since the introduction of novel disease modifying antirheumatic drugs (DMARDs). However, conventional DMARDs such as methotrexate, gold compounds, antimalarials, cyclosporine A, leflunomide, azathioprine, sulfasalazine and d-penicillamine are all associated with toxicity, and although methotrexate remains the standard of care in the United States and Europe for patients with RA, a response of less than 50%. improvement is often observed.

Initial data suggested that treatments that inhibit TNF-α have favorable toxicity profiles when compared to conventional DMARDS. However, long-term use of these treatments has resulted in concerns about toxicities such as an increased risk of infection (e.g., tuberculosis) and lymphoma, as well as an increased risk of systemic lupus erythematosus (Gabriel et al., A clinical and economic review of disease-modifying antirheumatic drugs. Pharmacoeconomics 19:715-28, 2001). Treatment with etanercept (ENBREL®), a currently available anti-TNF agent that is administered by injection, in combination with methotrexate has resulted in a clinical remission rate of 35% (Kremer, supra). However, the inconvenience of receiving injections and the side effects of anti-TNF therapy, including the development of serious infections as well as the inability to clear active infections, indicate that alternatives to conventional DMARD therapy and to anti-TNF agents are needed for the treatment of patients with RA (Kremer, supra).

The p38 mitogen-activated protein kinase (MAPK) pathway is involved in a number of cellular processes critical to the development of RA, such as upregulated expression of vascular cell adhesion molecule (VCAM) and intracellular adhesion molecule (ICAM) on endothelial cells, increased expression of MAC-1 and decreased expression of L-selectin on neutrophils, activation of Th1 lymphocytes; and upregulation of cytokine production by monocytes/macrophages. In addition, p38 MAPK regulates the differentiation of osteoclasts, which are directly involved in bone loss.

Thus, there is a need for additional therapies, dosage regimens and pharmaceutical compositions to treat RA. Specifically, there is a need for therapies, dosage regimens and pharmaceutical compositions comprising an inhibitor of p37 MAPK to treat RA.

### SUMMARY OF THE INVENTION

The present invention provides a method for treating RA, comprising administering VX-702, a p38 MAPK inhibitor, to a patient in need thereof.

In another embodiment, the invention provides a method for treating RA, comprising administering VX-702 and one or more other therapeutic agents useful for treating RA.

In another embodiment, the invention provides a pharmaceutical composition comprising VX-702 and a pharmaceutically acceptable carrier.

In another embodiment, the invention provides a pharmaceutical pack comprising VX-702 or a pharmaceutical composition thereof. In a further embodiment, the invention provides a pharmaceutical pack comprising VX-702, or a pharmaceutical composition thereof, and one or more other therapeutic agents useful for treating RA.

In another embodiment, the invention provides a kit comprising VX-702 or a pharmaceutical composition thereof and instructions for using VX-702 for treating RA.

### DETAILED DESCRIPTION OF THE INVENTION

The present invention relates to doses and methods of treating RA by administering VX-702, an orally available, specific and reversible inhibitor of the enzyme p38 MAPK. VX-702 has the following structure:

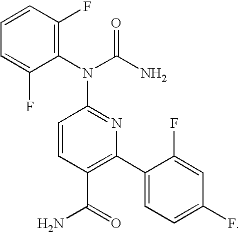

Five Phase 1 studies of VX-702 in healthy subjects and one Phase 2a study evaluating the safety and tolerability of VX-702 in subjects with unstable angina pectoris (UAP) who were scheduled for percutaneous coronary intervention with or without stenting were performed. Multiple dose studies of VX-702 were conducted for up to 28 days. Dosages of up to 20 mg/day were moderately to well tolerated in healthy subjects. Most adverse events were of mild or moderate severity and there were few severe events. Subjects with UAP were treated with up to 40 mg/day VX-702 for 5 days and adverse events were of mild to moderate severity.

In one embodiment, the invention provides pharmaceutical compositions comprising VX-702, or a pharmaceutically acceptable salt thereof, in an amount effective to treat RA, along with a pharmaceutically acceptable carrier.

In one embodiment, VX-702, or a pharmaceutically acceptable salt thereof, is provided in an amount from 1 to 20 mg in the pharmaceutical composition. In another embodiment, the invention provides a pharmaceutical composition comprising 2.5 to 15 mg VX-702, or a pharmaceutically acceptable salt thereof, in the pharmaceutical composition. In a further embodiment, the invention provides a pharmaceutical composition comprising 2.5 to 12.5 mg VX-702, or a pharmaceutically acceptable salt thereof, in the pharmaceutical composition. In yet a further embodiment, the invention provides a pharmaceutical composition comprising about 4, 5, 6, 7, 8, 9, 10 or 11 mg VX-702, or a pharmaceutically acceptable salt thereof, in the pharmaceutical composition. In yet a further embodiment, the invention provides a pharmaceutical composition comprising 4, 5, 6, 7, 8, 9, 10 or 11 mg VX-702, or a pharmaceutically acceptable salt thereof, in the pharmaceutical composition. In a further embodiment, the invention provides a pharmaceutical composition comprising about 5-10 mg VX-702, or a pharmaceutically acceptable salt thereof, in the pharmaceutical composition. In yet a further embodiment, the invention provides a pharmaceutical composition comprising 5-10 mg VX-702, or a pharmaceutically acceptable salt thereof, in the pharmaceutical composition. In a further embodiment, the invention provides a pharmaceutical composition of about 5 mg or 10 mg VX-702. In yet a further embodiment, the invention provides a pharmaceutical composition of 5 mg or 10 mg VX-702.

In another embodiment, the invention provides a pharmaceutical composition comprising about 1 mg to about 40 mg of VX-702, or a pharmaceutically acceptable salt thereof. In a further embodiment, the amount of VX-702, or a pharmaceutically acceptable salt thereof, is about 30 mg to about 40 mg of VX-702. In yet another embodiment, VX-702, or a pharmaceutically acceptable salt thereof, is present in an amount of about 20 mg to about 30 mg of VX-702. In another embodiment, the amount of VX-702, or a pharmaceutically acceptable salt thereof, is 2.5 mg, 5 mg, 7.5 mg, 10 mg, 12.5 mg, 15 mg, 20 mg, 25 mg, 30 mg, 35 mg or 40 mg.

As used herein, a recited specified amount or dose of VX-702 refers to that amount or dose of the free base form of VX-702. When “a pharmaceutically acceptable salt” of a specified amount of VX-702 is recited (e.g., “5 mg VX-702, or a pharmaceutically acceptable salt thereof”), the amount of the pharmaceutically acceptable salt is that quantity that is equivalent on a molar basis of VX-702 as the specified amount of the free base form of VX-702.

The amount of a pharmaceutically acceptable salt of VX-702 that is equivalent to a given quantity of the free base form of VX-702 is easily determined by one of skill in the art. One determines the molecular weight of the particular VX-702 salt form of interest and divides the molecular weight of this salt form by the molecular weight of the free base form of VX-702 to obtain the ratio of the weight of the salt to free base (salt/free base). Then, one multiplies the specified amount of the free base form by this ratio to obtain the equivalent amount of the VX-702 salt form.

Another embodiment of this invention provides a method for treating RA in a subject in need thereof comprising administering to the subject an effective amount of VX-702, or a pharmaceutically acceptable salt thereof. In general, VX-702, or a pharmaceutically acceptable salt thereof, is administered in a pharmaceutical composition comprising a pharmaceutically acceptable carrier.

As used herein, the terms “treat RA”, “treating RA”, or “treatment of RA” means lessening the severity of symptoms of RA. In one embodiment, the severity of symptoms of RA may be measured by physician and/or subject assessment of disease symptoms. These assessments may include, inter alia, reduction in number of swollen and/or tender joints, subject assessment of pain, subject self-assessment of disability, general health, physician and/or subject global assessments of disease, and/or acute phase response as measured by laboratory tests.

In one embodiment, assessment of disease symptoms, e.g., in clinical trials, is measured by the American College of Rheumatology (ACR) preliminary definition of improvement in RA (ACR₂₀).

In another embodiment, assessment of disease symptoms, e.g., in clinical trials, is measured by the European League Against Rheumatism (EULAR) response criteria. Other assessments of RA disease symptoms are also known and could be used to assess treatment of RA by VX-702.

In one embodiment, the amount of VX-702, or a pharmaceutically acceptable salt thereof, is between about 1 mg/day and 20 mg/day. In a particular embodiment, the amount of VX-702, or a pharmaceutically acceptable salt thereof, is at least about 1 mg/day. In another embodiment, the amount of VX-702, or a pharmaceutically acceptable salt thereof, is about 2.5 mg/day. In another embodiment, the amount of VX-702, or a pharmaceutically acceptable salt thereof, is about 4 mg/day. In another embodiment, the amount of VX-702, or a pharmaceutically acceptable salt thereof, is about 5 mg/day. In another embodiment, the amount of VX-702, or a pharmaceutically acceptable salt thereof, is about 6 mg/day. In another embodiment, the amount of VX-702, or a pharmaceutically acceptable salt thereof, is about 7 mg/day. In another embodiment, the amount of VX-702, or a pharmaceutically acceptable salt thereof, is about 8 mg/day. In another embodiment, the amount of VX-702, or a pharmaceutically acceptable salt thereof, is about 9 mg/day. In another embodiment, the amount of VX-702, or a pharmaceutically acceptable salt thereof, is about 10 mg/day. In another embodiment, the amount of VX-702, or a pharmaceutically acceptable salt thereof, is about 12.5 mg/day. In another embodiment, the amount of VX-702, or a pharmaceutically acceptable salt thereof, is about 15 mg/day. In another embodiment, the amount of VX-702, or a pharmaceutically acceptable salt thereof, is about 20 mg/day.

In yet another embodiment, the amount of VX-702, or a pharmaceutically acceptable salt thereof, is no more than about 20 mg/day. In another embodiment, the amount of VX-702, or a pharmaceutically acceptable salt thereof, is no more than about 15 mg/day. In another embodiment, the amount of VX-702, or a pharmaceutically acceptable salt thereof, is no more than about 12.5 mg/day. In another embodiment, the amount of VX-702, or a pharmaceutically acceptable salt thereof, is no more than about 10 mg/day. In another embodiment, the amount of VX-702, or a pharmaceutically acceptable salt thereof, is no more than about 5 mg/day. In another embodiment, the amount of VX-702, or a pharmaceutically acceptable salt thereof, is no more than about 2.5 mg/day.

It should be understood that these lower and upper amounts may be combined to provide preferred dose ranges for administering VX-702. For example, in one embodiment, VX-702, or the pharmaceutically acceptable salt thereof, is in an amount of about 2.5 mg to about 12.5 mg.

In any of these embodiments, the amount of VX-702 is administered once a day. Alternatively, the amount of VX-702 is administered twice a day (i.e., BID; q12 h) or three times daily (i.e., TID; q8 h).

In another embodiment, VX-702, or a pharmaceutically acceptable salt thereof, is administered once weekly, twice weekly, every third day or every other day. In a further embodiment, amount of VX-702, or a pharmaceutically acceptable salt thereof, administered once weekly, twice weekly, every third day or every other day is about 2.5 mg to about 40 mg per day. In yet a further embodiment, the amount of VX-702, or a pharmaceutically acceptable salt thereof, is about 2.5 mg to about 20 mg per day.

In a particular embodiment, the invention provides a method of treating RA in a subject, comprising administering 2.5-12.5 mg/day VX-702, or a pharmaceutically acceptable salt thereof, once daily to said subject. In another embodiment the invention provides a method of treating RA in a subject, comprising administering 5-10 mg/day VX-702, or a pharmaceutically acceptable salt thereof, once daily to said subject. In another embodiment, the invention provides a method of treating RA in a subject, comprising administering 5 or 10 mg/day VX-702, or a pharmaceutically acceptable salt thereof, once daily to said subject.

As described in greater detail in Example 1, VX-702 has been tested in humans and found to be effective for treating RA, i.e., for lessening the severity of symptoms of RA. Subjects receiving 5 mg or 10 mg of VX-702 once daily for twelve weeks showed an improvement in their symptoms compared to subjects receiving a placebo at Week 12 of treatment. Adverse events were generally mild to moderate.

Methods of this invention may also involve administration of one or more additional therapeutic agents for RA. Additional therapeutic agents that may be used with VX-702 include, without limitation, non-steroidal anti-inflammatory drugs (NSAIDS; e.g., aspirin, ibuprofen, naproxen, ketoprofen, indomethacin and celecoxib), local injection and/or oral administration of anti-inflammatory steroids (e.g., cortisone or prednisone), methotrexate, oral administration and/or intramuscular injections of gold compounds, antimalarials (e.g., hydroxychloroquine), cyclosporin, leflunomide, azathioprine, sulfasalazine, d-penicillamine, cyclophosphamide and mycophenolate; or combinations thereof. Accordingly, in another embodiment, this invention provides a method comprising administering VX-702 and one or more additional therapeutic agents for RA.

In one embodiment, an additional therapeutic agent that may be used with VX-702 is methotrexate. In a further embodiment, methotrexate is administered in an amount of about 1 to about 30 mg weekly. In yet a further embodiment, methotrexate is administered in an amount of about 2.5 to about 30 mg weekly. In yet a further embodiment, methotrexate is administered in an amount of about 5 to about 20 mg weekly. In yet a further embodiment, methotrexate is administered in an amount of about 5 to about 10 mg weekly. In another embodiment, methotrexate is administered twice weekly, once weekly, once every two weeks or once monthly. In a further embodiment, methotrexate is administered once weekly. In general, the methotrexate may be administered orally, as a pill or liquid formulation, or may be administered intravenously. One or more therapeutic agents in addition to VX-702 and methotrexate may also be used.

In another embodiment, additional therapeutic agents include biological agents. In a further embodiment, one or more biological agents are selected from a tumor necrosis factor α (TNFα) antagonist, an interleukin-1α (IL-1α) antagonist, a CD28 antagonist and a CD20 antagonist. In yet a further embodiment, one or more biological agents are selected from the group consisting of etanercept (ENBREL™), adalimumab (HUMIRA™), infliximab (REMICADE™), anakinra (KINERET™), abatacept (ORENCIA™), rituximab (RITUXAN™) and certolizumab pegol (CIMZIA™). One or more therapeutic agents in addition to VX-702 and one or more biological agents may also be used.

As is recognized by skilled practitioners, VX-702 is preferably administered orally. Some of the additional therapeutic agents for RA may be administered orally or may be administered in a different manner, such as intravenously, intramuscularly, parenterally, or via local injection into the site of inflammation. Nevertheless, nothing herein limits the methods or combinations of this invention to any specific dosage forms or regime. Thus, each component of a combination according to this invention may be administered separately, together, or in any combination thereof.

The methods herein may involve administration or co-administration of a) combinations of VX-702 and one or more other therapeutic agents for RA; or b) VX-702 in more than one dosage form. Co-administration includes administering each inhibitor in the same dosage form or in different dosage forms. When administered in different dosage forms, the inhibitors may be administered at different times, including about simultaneously or in any time period around administration of the other dosage forms. Separate dosage forms may be administered in any order. That is, any dosage forms may be administered prior to, together with, or following the other dosage forms.

VX-702, and any one or more additional therapeutic agents, may be formulated in separate dosage forms. Alternatively, to decrease the number of dosage forms administered to a patient, VX-702, and any additional agent(s), may be formulated together in any combination. Any separate dosage forms may be administered at the same time or different times. It should be understood that dosage forms should be administered within a time period such that the biological effects were advantageous.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” means any non-toxic salt that, upon administration to a recipient, is capable of providing a compound of this invention.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts.

This invention also envisions the quatemization of any basic nitrogen-containing groups of the compounds disclosed herein. Water or oil-soluble or dispersable products may be obtained by such quatemization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds utilized in the compositions and methods of this invention may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

According to a preferred embodiment, the compositions of this invention are formulated for pharmaceutical administration to a mammal, particularly a human being.

Such pharmaceutical compositions of the present invention (as well as compositions for use in methods, combinations, kits, and packs of this inventions) may be administered orally, parenterally, sublingually, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. More preferably, the compositions are administered orally.

Sterile injectable forms of the compositions of and according to this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In compositions of this invention comprising VX-702 and one or more additional therapeutic agents, VX-702 and the additional agent should be present at dosage levels of between about 10 to 100%, and more preferably between about 10 to 80% of the dosage normally administered in a monotherapy regimen.

The pharmaceutical compositions of this invention may be orally administered in any orally acceptable dosage form including, but not limited to, capsules, tablets, pills, powders, granules, aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. Acceptable liquid dosage forms include emulsions, solutions, suspensions, syrups, and elixirs.

Alternatively, the pharmaceutical compositions of this invention may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically.

As is recognized in the art, pharmaceutical compositions may also be administered in the form of liposomes.

Applicants have demonstrated that VX-702 is orally bioavailable. Accordingly, preferred pharmaceutical compositions of this invention are formulated for oral administration.

Administration of VX-702 in connection with this invention can be used as a chronic or acute therapy. The amount of active ingredient that may be combined with the carrier materials to produce a single dosage form will vary depending upon the host treated and the particular mode of administration. A typical preparation will contain from about 0.5% to about 95% active compound (w/w). Preferably, such preparations contain from about 5% to about 90% active compound.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

Pharmaceutical compositions may also be prescribed to the patient in “patient packs” or “pharmaceutical packs” containing the whole course of treatment in a single package, usually a blister pack. Patient packs have an advantage over traditional prescriptions, where a pharmacist divides a patients supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination of the invention by means of a single patient pack, or patient packs of each formulation, containing within a package insert instructing the patient to the correct use of the invention is a desirable additional feature of this invention.

According to a further aspect of the invention is a pack comprising at least VX-702 (in dosages according to this invention) and an information insert containing directions on the use of the combination of the invention. Any composition, dosage form, therapeutic regimen or other embodiment of this invention may be presented in a pharmaceutical pack. In an alternative embodiment of this invention, the pharmaceutical pack further comprises one or more additional therapeutic agents as described herein. The additional therapeutic agent or agents may be provided in the same pack or in separate packs. In one embodiment, the additional therapeutic agent is methotrexate.

Another aspect of this involves a packaged kit for a patient to use in the treatment of RA, comprising: a single or a plurality of pharmaceutical formulations of VX-702; a container housing the pharmaceutical formulation(s) during storage and prior to administration; and instructions for carrying out drug administration in a manner effective to treat RA.

In another embodiment, the packaged kit further comprises one or more pharmaceutical formulations comprising additional therapeutic agents useful for treating RA. Accordingly, this invention provides kits for the simultaneous or sequential administration of a dose of VX-702 and one or more additional therapeutic agent. Typically, such a kit will comprise, e.g. a composition of each compound and optional additional agent(s) in a pharmaceutically acceptable carrier (and in one or in a plurality of pharmaceutical formulations) and written instructions for the simultaneous or sequential administration. In one embodiment, the additional therapeutic agent is methotrexate.

In another embodiment, a packaged kit is provided that contains one or more dosage forms for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle. The present kits will also typically include means for packaging the individual kit components, i.e., the dosage forms, the container means, and the written instructions for use. Such packaging means may take the form of a cardboard or paper box, a plastic or foil pouch, etc.

A kit according to this invention could embody any aspect of this invention such as any composition, dosage form, therapeutic regimen, or pharmaceutical pack.

The packs and kits according to this invention optionally comprise a plurality of compositions or dosage forms. Accordingly, included within this invention would be packs and kits containing one composition or more than one composition.

Although certain exemplary embodiments are depicted and described below, it will be appreciated that compounds of this invention can be prepared according to the methods described generally above using appropriate starting materials generally available to one of ordinary skill in the art.

In order that this invention be more fully understood, the following example is set forth. This example is for the purpose of illustration only and is not to be construed as limiting the scope of the invention in any way.

### EXAMPLE 1

VX-702 was examined in a randomized, placebo-controlled, multiple-dose, blinded, dose escalation study in 315 subjects with moderate or severe RA.

Subjects were divided into 3 approximately equal-sized groups of 100-105 subjects each. In one group, subjects received 5 mg VX-702 once daily or 2.5 mg VX-702 twice daily (total 5 mg/day) for twelve weeks. Another group received 10 mg VX-702 once daily for twelve weeks. Another group subjects received placebo once daily for twelve weeks.

The compositions used in study were as follows:

| | 2.5 mg tablet | 10 mg tablet |
|---|---|---|
| Reagent | % by weight | % by weight |
| VX-702 | 2.5 | 10 |
| Lactose Monohydrate, NF (Foremost#310) | 10 | 10 |
| Lactose Monohydrate, NF (Fast-Flo, #316) | 53.75 | 46.25 |
| Dibasic Calcium Phosphate (Emcompress) | 16 | 16 |
| Microcrystalline Cellulose, USP | 15 | 15 |
| (Avicel PH 101) | | |
| Sodium Lauryl Sulphate, NF | 0.5 | 0.5 |
| Croscarmellose Sodium, NF | 1 | 1 |
| (AcDisol SD-711) | | |
| Colloidal Silicon Dioxide, NF | 0.25 | 0.25 |
| (Cab-O-Sil M5P) | | |
| Magnesium Stearate, NF | 1 | 1 |

The subjects had to be between the ages of 18-75 years (inclusive) and have active RA of six months of duration or longer by the ACR revised criteria. The subjects had to have a C-reactive protein (CRP) serum levels of greater than 2 mg/dL at time of randomization, a swollen joint count of eight or greater (of 28) and tender joint count of 10 or greater (of 28). The subjects had to have had no prior treatment with Disease modifying anti-rheumatic drugs (DMARD) therapy or inadequate response to DMARD therapy. If a subject had received prior therapy with an antibody or binding protein to TNF (anti-TNF) or with recombinant IL-1 receptor antagonist (IL-1Ra), the subject may have discontinued treatment due to tolerability reasons but not have discontinued treatment because of an inadequate response. Subjects must also have discontinued DMARD therapy (except for sulfasalazine or hydroxychloroquine) for at least one month prior to randomization. Subjects could receive one NSAID and/or prednisone (≦10 mg/day) if they had been treated at a stable dose for at least one month prior to randomization.

Safety evaluations of subjects in the study were also conducted. These included physical examinations, including vital signs measurements (including blood pressure, heart rate, respiration rate, and temperature); clinical laboratory assessments (hematology, chemistry, and urinalysis testing); creatinine clearance; adverse events; Holter monitoring and electrocardiogram (ECG) (12-lead).

Responses to treatment were evaluated by changes in ACR (e.g., ACR₂₀) or EULAR criteria. Responses to treatment were measured at Weeks 2, 4, 6, 8, 10 and 12. Safety evaluations were also conducted at these time points. Holter monitoring and ECG were not performed at every time point. Four weeks after completion of the study, responses to treatment and safety evaluations were conducted on subjects.

ACR Preliminary Definition of Improvement in RA (ACR₂₀):

Required: ≧20% improvement in tender joint count
≧20% improvement in swollen joint count And, at least 20% improvement in 3 of the following 5:
Subject pain assessment
Subject global assessment
Physician global assessment
Subject self-assessed disability

Acute-phase reactant (CRP or ESR)

| Disease activity measure | Method of assessment |
|---|---|
| Tender joint count | ACR tender joint count, an assessment of 28 joints. The |
| | joint count should be done by scoring several different |
| | aspects of tenderness, as assessed by pressure and joint |
| | manipulation on physical examination. The information |
| | on various types of tenderness should then be collapsed |
| | into a single tender-versus-nontender dichotomy. |
| Swollen joint count | ACR swollen joint count, an assessment of 28 joints. |
| | Joints are classified as either swollen or not swollen. |
| Subject pain assessment | A horizontal pain VAS (0-100 mm) is used to assess |
| | the subject's current level of pain. |
| Subject global assessment | The subject's overall assessment of their arthritis is |
| | documented on a 0-10 scale. |
| Physician global assessment | The physician's assessment of the subject's disease |
| | activity is documented on a 0-10 scale |
| Subject self-assessed disability | The HAQ self-assessment instrument which measures |
| | physical function in RA subjects is acceptable, |
| | validated, has reliability, and has been proven in RA |
| | trials to be sensitive to change. |
| Acute-phase reactant | A Westergren erythrocyte sedimentation rate or C- |
| | reactive protein level |

The ACR₅₀ and ACR₇₀ responses require ≧50% and ≧70% improvement, respectively, in the same criteria as described for the ACR₂₀ response.

EULAR Response Criteria

EULAR is described in Fransen et al., Clinical and Experimental Rheumatology 23 (Suppl. 39): S93-99, 2005, which is hereby incorporated by reference in its entirety. EULAR is based on the Disease Activity Score (DAS), a clinical index of RA disease activity that combines information from swollen joints, tender joints, the acute phase response and general health into one continuous measure of rheumatoid inflammation. DAS₂₈ is an index similar to the original DAS, consisting of a 28 tender joint count (range 0-28), a 28 swollen joint count (range 0-28), erythrocyte sedimentation rate (ESR) and an optional general health assessment on a visual analogue scale (range 0-100).

Safety Results

VX-702 was well tolerated with treatment discontinuation rate for adverse events being low and similar to that seen in the placebo group. Premature discontinuations for adverse events occurred in 2% of patients receiving placebo, 3% of the 5 mg VX-702 patients, and 5% of the 10 mg VX-702 patients. The most common adverse events that led to treatment discontinuation were seen in two patients each and were as follows: gastroenteritis, nausea/vomiting, rash and renal impairment (increased serum creatinine). No patients discontinued for elevations in liver function tests. Isolated serious adverse events were reported in 2% of placebo-treated patients, and 4-7% of VX-702-treated patients. Gastroenteritis was the only serious adverse event reported in more than one patient.

The most common adverse events were generally rated mild or moderate and were infections (upper respiratory infections, gastroenteritis, nasopharyngitis, etc), seen in 10% of VX-702-treated patients versus 5% of placebo recipients; gastrointestinal disorders (nausea, vomiting, diarrhea) seen in 8% of VX-702-treated patients versus 6% of placebo recipients; and skin disorders (rash, acne, itching) seen in approximately 9% of VX-702-treated patients.

No clinically significant effects on laboratory parameters, including liver function tests, were evident. At each on-treatment visit, 2-4% of VX-702-treated patients and 1-2% of placebo recipients exhibited an ALT value that was above the upper limit of normal. No patient developed an ALT elevation to three times the upper limit of normal, which would have required treatment discontinuation.

Extensive Holter (24 to 72-hour continuous electrocardiogram (EKG) monitoring conducted 6 times/patient during the study) did not did not reveal any differences in the rates of ventricular ectopic activity between patients receiving placebo and those receiving VX-702, and did not reveal an increased tendency for arrhythmias with VX-702 treatment. Digital electrocardiograms revealed a minimal increase in the QT interval: from a baseline QTcF (Fridericia-corrected QT) of approximately 400 msec at the end of treatment placebo patients demonstrated a mean −0.6 msec change, while the VX-702 patients exhibited mean 3 and 6 msec increase in QTcF in the 5 and 10 mg groups, respectively. No patient demonstrated a maximal increase in QTcF of >60 msec at any point in the study.

Effects on Signs and Symptoms of Rheumatoid Arthritis

Final analysis of the study results demonstrated there was a dose-dependent statistically significant increase in ACR20 response rates with VX-702 treatment: 28% of placebo recipients, 36% of 5 mg VX-702-treated patients, and 40% of VX-702-treated patients achieving an ACR20 response (p=0.02; Jonckheere-Terpstra test for increasing dose-response). In addition, 32% of placebo recipients, 40% of 5 mg 702-treated patients, and 44% of 10 mg VX-702 treated patients achieved an EULAR (moderate or good) response (p=0.02). Dose-dependent statistically significant effects were also demonstrated percent improvement in DAS28. ACR20, EULAR, DAS28, percent change in tender joints, percent change in swollen joints and reduction in morning stiffness are presented in the table below:

| | End of Treatment (Week 12) | | | |
|---|---|---|---|---|
| | | | 10 mg | |
| | Placebo | 5 mg VX-702 | VX-702 | p value |
| ACR 20 Response | 28% | 36% | 40% | 0.02 |
| EULAR Response | 32% | 40% | 44% | 0.02 |
| Improvement from | −0.8 | −1.0 | −1.2 | 0.012 |
| baseline in DAS28 | | | | |
| Percent Improvement | −12% | −15% | −18% | 0.009 |
| in DAS | | | | |
| Percent Change in | −22% | −28% | −36% | ND |
| Tender Joints | | | | |
| Percent Change in | −28% | −35% | −46% | ND |
| Swollen Joints | | | | |
| Mean reduction in | −3.0 | −98 | −82 | <0.001 |
| morning stiffness | | | | |
| (minutes) | | | | |
| Percent reduction in | 7.2% | −21.6% | −2.7% | <0.001 |
| morning stiffness | | | | |

All cited documents are incorporated herein by reference.

While we have described a number of embodiments of this invention, it is apparent that our basic examples may be altered to provide other embodiments which utilize the compounds and methods of this invention. Therefore, it will be appreciated that the scope of this invention is to be defined by the appended claims rather than by the specific embodiments that have been represented by way of example above.

1. A pharmaceutical composition comprising VX-702, or a pharmaceutically acceptable salt thereof, in an amount effective to treat rheumatoid arthritis, and a pharmaceutically acceptable carrier.

2. The pharmaceutical composition according to claim 1, comprising VX-702, or a pharmaceutically acceptable salt thereof, in an amount of about 1 mg to about 20 mg of VX-702.

3-8. (canceled)

9. The pharmaceutical composition according to claim 1, comprising VX-702, or a pharmaceutically acceptable salt thereof, in an amount of about 1 mg to about 40 mg of VX-702.

10-11. (canceled)

12. A method of treating rheumatoid arthritis in a subject in need thereof, comprising administering VX-702, or a pharmaceutically acceptable salt thereof, in an amount effective to treat said rheumatoid arthritis in said patient.

13. (canceled)

14. The method according to claim 12, comprising administering the subject VX-702, or a pharmaceutically acceptable salt thereof, in an amount of about 1 mg to about 20 mg per day.

15-20. (canceled)

21. The method according to claim 12, comprising administering the subject VX-702, or a pharmaceutically acceptable salt thereof, in an amount of about 1 mg to about 40 mg per day.

22-37. (canceled)

38. The method according to claim 12, wherein said method comprises administering one or more additional therapeutic agents for rheumatoid arthritis.

39. The method according to claim 38, wherein said one or more additional therapeutic agents are selected from the group consisting of a non-steroidal anti-inflammatory drug, an anti-inflammatory steroid, methotrexate, a gold compound, an antimalarial, cyclosporin, leflunomide, azathioprine, sulfasalazine, d-penicillamine, cyclophosphamide and mycophenolate.

40. The method according to claim 39, wherein said one or more additional therapeutic agents is methotrexate.

41-49. (canceled)

50. The method according to claim 38, wherein said one or more additional therapeutic agents are selected from one or more biological agents.

51. The method according to claim 50, wherein said one or more biological agents are selected from the group consisting of a tumor necrosis factor α(TNFα) antagonist, an interleukin-1α(IL-1α) antagonist, a CD28 antagonist and a CD20 antagonist.

52. The method according to claim 51, wherein said one or more biological agents are selected from the group consisting of etanercept (ENBREL™), adalimumab (HUMIRA™), infliximab (REMICADE™), anakinra (KINERET™), abatacept (ORENCIA™), rituximab (RITUXAN™) and certolizumab pegol (CIMZIA™).

53. A pharmaceutical pack or kit comprising a composition comprising VX-702 according to claim 1, or a composition or dosage form comprising VX-702 for practicing the method according to claim 12.

54. The pharmaceutical pack or kit according to claim 53, wherein said pharmaceutical pack comprises VX-702 or a pharmaceutical composition thereof, and methotrexate or a pharmaceutical composition thereof.

55-60. (canceled)

61. The pack or kit according to claim 53, comprising a plurality of compositions or dosage forms comprising VX-702 or a pharmaceutically acceptable salt thereof.

62. The pack or kit according to claim 54, comprising a plurality of compositions or dosage forms comprising VX-702 or a pharmaceutical composition thereof, and a plurality of compositions or dosage forms comprising methotrexate or a pharmaceutical composition thereof.

63-64. (canceled)

65. A pharmaceutical composition comprising VX-702.

66. The composition according to claim 65, wherein said composition comprises about 1 to about 40 mg VX-702, about 10 to about 20% dibasic calcium phosphate, about 10 to about 20% microcrystalline cellulose, about 0.1 to about 1.0% sodium lauryl sulfate, about 0.1 to about 2.5% croscarmellose sodium, about 0.0 to about 1% colloidal silicon dioxide, about 0.1 to about 5% magnesium stearate and about 10 to about 70% lactose monohydrate.
